(19) United States
(12) Patent Application Publication
(10) Pub. No.: US 2008/0039722 A1
(43) Pub. Date: Feb. 14, 2008

(54) SYSTEM AND METHOD FOR PHYSIOLOGICAL SIGNAL EXCHANGE BETWEEN AN EP/HEMO SYSTEM AND AN ULTRASOUND SYSTEM

(75) Inventors: Claudio Patricio Mejia, Wauwatosa, WI (US); Richard William Schefelker, Menomonee Falls, WI (US); Zvi Geffen, Ramat Begin (IL)

Correspondence Address: MCANDREWS HELD & MALLOY, LTD, 500 WEST MADISON STREET, SUITE 3400, CHICAGO, IL, 60661, US

(73) Assignee: GENERAL ELECTRIC COMPANY, Schenectady, NY (US)

(21) Appl. No.: 11/620,896

(22) Filed: Jan. 8, 2007

Related U.S. Application Data
(60) Provisional application No. 60/822,210, filed on Aug. 11, 2006.

(51) Int. Cl. A61B 8/00; A61B 5/00

(52) U.S. Cl. 600/437; 600/301

(57) ABSTRACT

Certain embodiments provide systems and methods for exchanging physiological signal data between an EP/Hemo system and an ultrasound system in an integrated EP/Hemo and ultrasound system environment. Certain embodiments of a method include obtaining physiological signal data; communicating the physiological signal data between the EP/Hemo system and the ultrasound system via signal communication ports; displaying the physiological signal data at both of the EP/Hemo system and the ultrasound system; and correlating the physiological signal data with image data from the ultrasound system. Certain embodiments of a system include an EP/Hemo system having a signal port, an ultrasound system having a signal port, a connection between the EP/Hemo system and the ultrasound system, and physiological signal data being communicated.

### RELATED APPLICATIONS

This application claims priority to a provisional application entitled “System and Method for Physiological Signal Exchange Between an EP/Hemo System and an Ultrasound System,” filed on Aug. 11, 2006, as Ser. No. 60/822,210, which is herein incorporated by reference in its entirety.

### BACKGROUND OF THE INVENTION

The present invention generally relates to an integrated electrophysiology/hemodynamic (EP/Hemo) and ultrasound environment. More specifically, certain embodiments of the present invention related to systems and methods for exchanging physiological signal data between an EP/Hemo system and an ultrasound system in an integrated EP/Hemo and ultrasound system environment.

During clinical invasive procedures, such as interventional cardiology or radiology procedures, there is a need to continuously monitor physiological parameters of a patient. Monitoring a patient is done using physiological monitoring and recording systems, such as the GE Mac-Lab for hemodynamic procedures and the GE CardioLab for electrophysiology procedures.

Hemodynamic monitoring can aid in detection, identification, and treatment of life-threatening conditions such as heart failure and cardiac tampanade. Using invasive hemodynamic monitoring, for example, a practitioner can help evaluate a patient's response to treatment, such as drugs and mechanical support. A practitioner can evaluate the effectiveness of cardiovascular function such as cardiac output and cardiac index.

Electrophysiological data includes an analysis of the electrical conduction system of a patient's heart, which generates a heart beat. Catheters may be insert in a vein are then passed into the heart under fluoroscopic guidance, for example. The catheters measure the electrical signals generated by the heart to obtain a more detailed analysis of the electrical signals than a simple electrocardiogram (ECG).

Invasive and/or noninvasive techniques can be used to determine hemodynamic and/or electrophysiological data for a patient. For example, a patient's blood pressure may be measured using a cuff, and/or pressure with a heart may be measured invasively using a catheter. Blood and/or heart pressure measurement may include a systolic pressure and a diastolic pressure. Using the two measurements, a mean pressure can be calculated. Parameters such as chest cardiac output (CO), cardiac index (CI), pulmonary artery wage pressures (PAWP), and cardiac index (CI) may be measuring using a catheter.

Currently, ultrasound systems can receive waveform data from signal acquisition devices but not in the context of an integrated EP/Hemo and ultrasound environment. Additionally, waveform data has typically been acquired and later provided to an ultrasound system. Furthermore, current systems do not allow real-time or substantially real-time use of physiological waveform data with ultrasound imaging data in an EP/Hemo or ultrasound system.

Thus, there is a need for systems and methods for integrating EP/Hemo and ultrasound systems. There is a need for systems and methods for exchanging waveform and other physiological data in an integrated EP/Hemo and ultrasound environment.

### BRIEF SUMMARY OF THE INVENTION

Certain embodiments provide systems and methods for exchanging physiological signal data between an electrophysiologic/hemodynamic (EP/Hemo) system and an ultrasound system in an integrated EP/Hemo and ultrasound system environment. Certain embodiments of a method include obtaining physiological signal data; communicating the physiological signal data between the EP/Hemo system and the ultrasound system via signal communication ports; and displaying the physiological signal data at both of the EP/Hemo system and the ultrasound system. In certain embodiments, the method may also include correlating the physiological signal data with image data from the ultrasound system.

Certain embodiments of an integrated EP/Hemo and ultrasound system include an EP/Hemo system for obtaining physiological signal data for a patient. The EP/Hemo system includes a first signal communication port. The integrated system also includes an ultrasound system for obtaining ultrasound data for the patient. The ultrasound system includes a second signal communication port. The integrated system further includes a communication channel between the EP/Hemo system and the ultrasound system for communicating the physiological signal data between the EP/Hemo system and the ultrasound system via the first and second signal communication ports. The EP/Hemo system and the ultrasound system are configured for display of the physiological signal data at both of the EP/Hemo system and the ultrasound system.

Certain embodiments of a method for integrating physiological and imaging data for a patient include providing physiological waveform data for a patient; providing image data for the patient; correlating the physiological waveform data and the image data for the patient; and displaying the physiological signal waveform data and the image data in an integrated physiological and imaging environment.

### BRIEF DESCRIPTION OF SEVERAL VIEWS OF THE DRAWINGS

FIG. 1 illustrates a high-level system diagram of an integrated EP/Hemo and ultrasound system in accordance with an embodiment of the present invention.

FIG. 2A illustrates a diagram of an integrated EP/Hemo and ultrasound system in accordance with an embodiment of the present invention.

FIG. 2B illustrates a diagram of an integrated EP/Hemo and ultrasound system in accordance with an embodiment of the present invention.

FIG. 3 illustrates a flow diagram for a method for exchanging physiological signal data between an EP/Hemo recording/monitoring system and an ultrasound system in accordance with an embodiment of the present invention.

The foregoing summary, as well as the following detailed description of certain embodiments of the present invention, will be better understood when read in conjunction with the appended drawings. For the purpose of illustrating the invention, certain embodiments are shown in the drawings. It should be understood, however, that the present invention is not limited to the arrangements and instrumentality shown in the attached drawings.

### DETAILED DESCRIPTION OF THE INVENTION

Certain embodiments of the present invention provide an electrophysiology and hemodynamic (EP/Hemo) recording or monitoring system with an ability to exchange physiological signal data with an ultrasound system. Certain embodiments provide methods for exchanging physiological signal data between an EP/Hemo recording system and an ultrasound system or other similar system. The signal data may be electrocardiogram (ECG) and/or intracardiac waveform data, for example. In certain embodiments, EP/Hemo recording systems are tightly integrated with ultrasound systems. Integration allows information, such as physiological waveform information, to be exchanged. The waveform information is thus available both in the EP/Hemo System and in the ultrasound system (e.g., the GE Vivid-I system) simultaneously or substantially simultaneously (e.g., concurrently with some inherent delay). Both systems are displaying the same signal, which allows signal information to be correlated with image information (e.g., correlation of ECG/intracardiac waveform data with an ultrasound image).

Certain embodiments of the EP/Hemo data acquisition system provide an analog physiological signal to the ultrasound system. The connection from the EP/Hemo data acquisition system to the ultrasound system may be implemented using an ECG or other waveform input port, for example. In an embodiment, an ECG input port is used with a two-wire cable (one wire for an ECG electric signal and one wire for ground) having an input level of plus or minus 1 V, an input impedance of greater than 10 megaohms, and a source-dependent bandwidth and dynamic range. Details of the port and connecting cable may vary greatly depending upon system, data and operating conditions. This is but one illustrative example.

In certain embodiments, waveform data may be provided “as-is” to an ultrasound system. Alternatively and/or in addition, waveform data may be processed and/or otherwise prepared for display by the EP/Hemo system before being provided to the ultrasound system, for example. In certain embodiments, waveform data may be displayed by the ultrasound system, used to gate image capture, used to assist in performing image processing, etc.

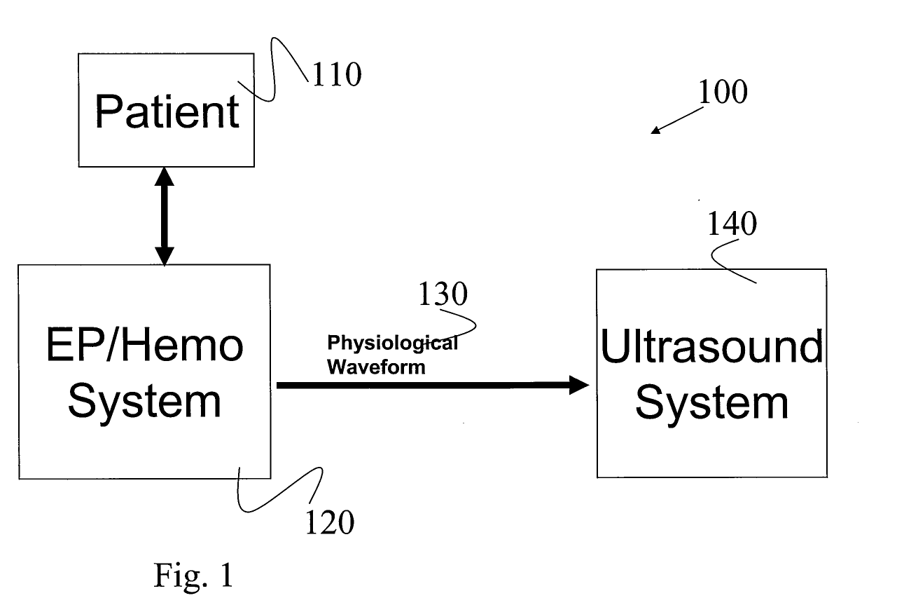

FIG. 1 illustrates a high-level system diagram of an integrated EP/Hemo and ultrasound system 100 in accordance with an embodiment of the present invention. The system 100 includes a patient providing data 110 to an EP/Hemo system 120. The EP/Hemo system 120 sends physiological waveform data 130 and/or other data to an ultrasound system 140 (e.g., a Vivid-I or other ultrasound system).

The EP/Hemo system 120 obtains EP and/or hemo data for one or more patients. In order to share information, the EP/Hemo system 120 may provide one or more interfaces to different hemodynamic systems, electrophysiological systems, catheterization lab systems, and database systems, for example. The information can be collected before, during and/or after a catheterization procedure and may be shared with laboratory and hospital repository systems (e.g., orders and results) for a patient record. Interface(s) may be based on industry-standard protocols (e.g., HL7, SQL, ASCII) and/or specific interface(s) for systems that do not support standard protocols, for example. The interface(s) allow exchange and sharing of data (e.g., demographics, history, log, results etc.) between different systems and vendors, for example.

The EP/Hemo system 120 can combine hemodynamic and electrophysiological monitoring into a single system configuration to allow dual use of a catheterization or other lab. EP and hemo data can be stored in a single database to help streamline documentation and access to patient information. The EP/Hemo system 120 provides laboratory performance and resources for patient care. In certain embodiments, the EP/Hemo system 120 may be used in one or more locations, as well as in transit, for example. In certain embodiments, the EP/Hemo system 120 may be accessed remotely.

In certain embodiments, the EP/Hemo system 120 includes a graphical user interface to facilitate user-defined procedural lists, macros and configurable electronic documentation. The EP/Hemo system 120 may include a multi-parameter module, such as a GE TRAM® module, that acquires and processes patient physiological parameters, such as ECG, invasive blood pressure, non-invasive blood pressure, pulse oximetry, cardiac output, temperature, respiration, etc. Patient data may be measured in real-time and/or substantially real-time, for example. The EP/Hemo system 120 may also be configured for administrative reporting and facilitation of clinical workflow. The EP/Hemo system 120 may further provide on-line help resources and an ability to save data to a network and/or attached storage, for example.

The EP/Hemo system 120 may include a variety of inputs/outputs, such as one or more ECG leads, one or more stimulation inputs, one or more invasive pressure signals, one or more recording channels, one or more intracardiac channels, one or more catheter inputs, etc. The EP/Hemo system 120 provides diagnostic tools, as well as intracardiac and ECG recording capability, for example. In certain embodiments, the system 120 provides bi-polar channel scalability, automated clinical features and activation mapping to aid in diagnosis. The system 120 may provide a 3D mapping interface as well as connectivity to external system(s), for example. In certain embodiments, the EP/Hemo system 120 may interface uni- or bi-directionally with another system, such as a navigation and/or ablation system to share information, such as mapping events, clinical data and/or EP report data. The EP/Hemo system 120 may be configured to operate in a plurality of languages.

In certain embodiments, the ultrasound system 140 may be configured to provide one or more data acquisition modes and/or data processing capabilities, for example. The system 140 may include one or more probes, such as phased array sector probes, linear array probes, convex array (curved) probes, Doppler pencil probes, multiplane transesophageal phased array probes, etc. The system 140 provides imaging in one or more modes such as 2D mode, M mode, anatomical M mode, color Doppler, color angio, color M mode, anatomical color M mode, spectral Doppler, Pulsed Wave/High Pulse Repetition Frequency (PW/HPRF) Doppler, Tissue Doppler, CW Doppler, etc.

The ultrasound system 140 may be configured for a variety of data processing. The system 140 may provide echo data processing of phase, amplitude and frequency, for example. The system 140 may provide digital raw data replay for image post post-processing and offline measurement and analysis. The system 140 may include an instant review screen to display one or more loops/images for study review. In certain embodiments, a scan plane position indicator and probe temperature may be displayed with multi-plane transesophageal probes. An image orientation indicator may be displayed with image data.

A display integrated with and/or associated with the ultrasound system 140 may be configured for a plurality of views including single, dual and quad-screen view. In certain embodiments, the system 140 provides a selectable display configuration of duplex and triplex modes either side-by-side or top-bottom.

In certain embodiments the ultrasound system 140 provides variable transmit frequencies for resolution/penetration improvement. The system 140 may also provide variable contour filtering for edge enhancement.

The ultrasound system 140 may also provide a variety of analysis and workflow tools. For example, personalized measurement protocols allow individual setting and ordering of measurement and analysis items. Measurements may be labeled using protocols and/or post assignments, for example. Bodymark icons may be provided for location and position of a probe. In certain embodiments, the system 140 provides cardiac calculation and/or vascular measurement functionality including measurement and display of multiple, repeated measurements. In certain embodiments, measurements are assignable to one or more protocols and/or report generators. Parameter(s) and/or parameter annotation may follow a medical standard, such as an American Society of Echocardiography standard, and/or may be user-assignable, for example. Certain embodiments provide a Doppler auto-trace function including automatic calculation in live and/or digital replay, for example. Functions, such as data storage and report creation, may be combined and/or automated in a variety of ways, for example.

In certain embodiments, the ultrasound system 140 and/or the EP/Hemo system 120 may access a knowledge database and/or guidance center, such as GE's iLinq™ system, for system-specific and/or context-sensitive support. The system 120 and/or system 140 may also communicate with a remote diagnostic and support center, such as GE's InSite™.

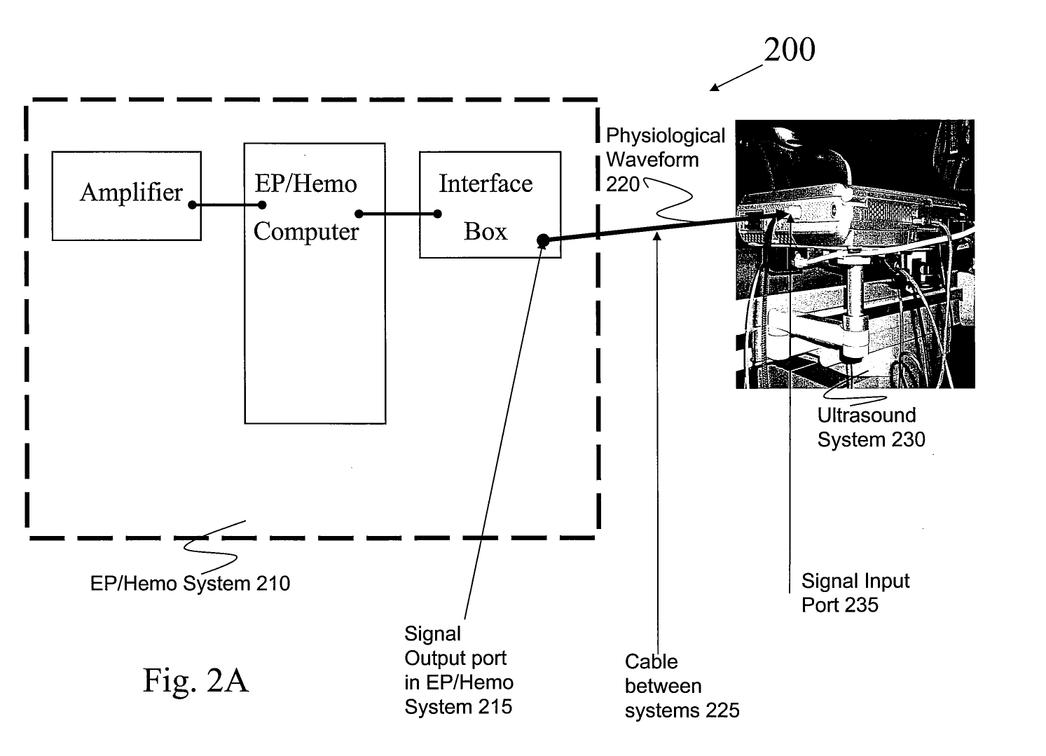

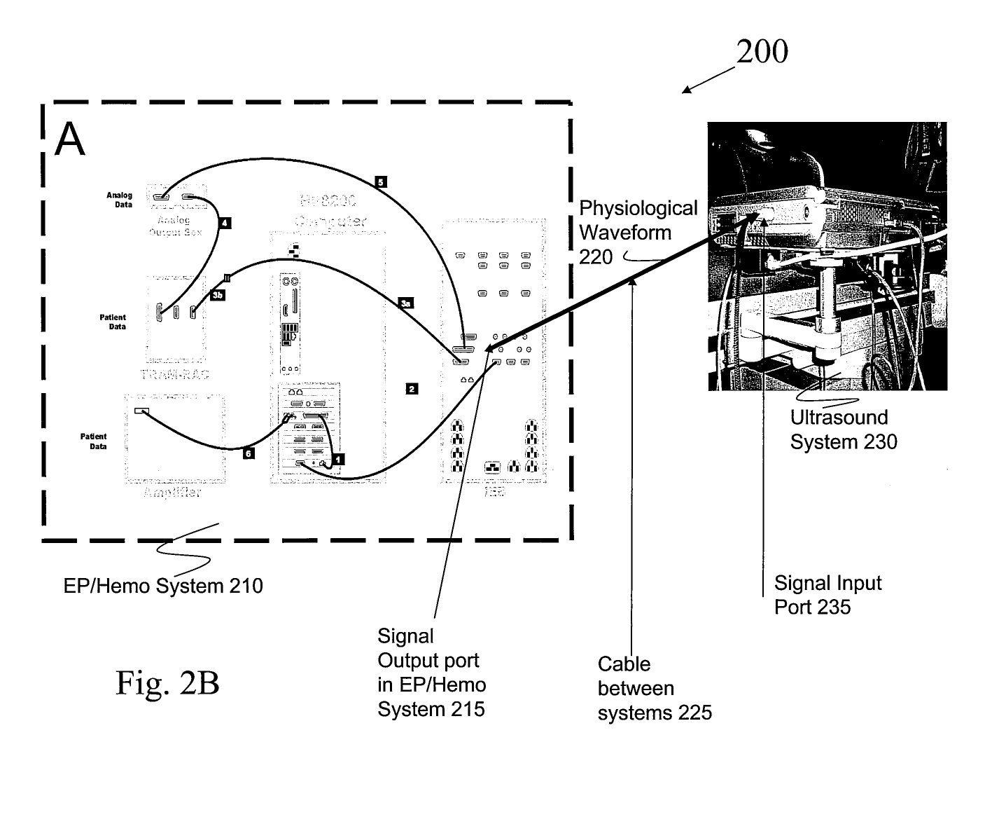

FIG. 2A illustrates a diagram of an integrated EP/Hemo and ultrasound system 200 in accordance with an embodiment of the present invention. The system 200 includes an EP/Hemo system 210, physiological waveform data 220 and/or other data on a cable 225, and an ultrasound system 230. The ultrasound system 230 includes a signal input port 235. The EP/Hemo system 210 also includes a signal output port 215, among other components.

As described above, data, such as physiological waveform data 220, is acquired from a patient or external system via the EP/Hemo system 210. The data 220 is transmitted from the EP/Hemo system 210 via the signal output port 215. The waveform data 220 is transmitted to the ultrasound system 230 via the cable connection 220. Note that the cable connection 220 may encompass a variety of cable connections, as well as non-cable connections such as wireless, infrared, etc. The data 220 is received at the signal input port 235 of the ultrasound system 230. Similarly, data 220 may be communicated from the ultrasound system 230 to the EP/Hemo system 210 via the connection 225 and ports 235, 215.

As shown in FIG. 2A, the EP/Hemo system 210 may include an amplifier for patient waveform data amplification. The system 210 may also include an EP/Hemo computer for processing and/or storage waveform and/or other data, for example. The system 210 may also include an interface for facilitating communication and/or data transfer between the system 210 and the ultrasound system 230 via the connection 225.

The waveform data 220 may be displayed and/or used in diagnosis and/or reporting at the EP/Hemo system 210 and/or ultrasound system 230, for example. The waveform data 220 may be correlated with image data from the ultrasound system 230 for processing and/or display, for example.

FIG. 2B illustrates another diagram of an integrated EP/Hemo and ultrasound system 200 in accordance with an embodiment of the present invention. The system 200 includes an EP/Hemo system 210, physiological waveform data 230 and/or other data on a cable 225, and an ultrasound system 230. The ultrasound system 230 includes a signal input port 235. The EP/Hemo system 210 also includes a signal output port 215, among other components.

As described above in relation to FIGS. 1 and 2A, data, such as physiological waveform data 220, is acquired from a patient or external system at the EP/Hemo system 210. The data 220 is transmitted from the EP/Hemo system 210 via the signal output port 215. The waveform data 220 is transmitted to the ultrasound system 230 via the cable connection 220. Note that the cable connection 220 may encompass a variety of cable connections, as well as non-cable connections such as wireless, infrared, etc. The data 220 is received at the signal input port 235 of the ultrasound system 230. Similarly, data 220 may be communicated from the ultrasound system 230 to the EP/Hemo system 210 via the connection 225 and ports 235, 215.

As discussed above in relation to FIG. 2A, the EP/Hemo system 210 may include a computing device, an interface, and/or an amplifier, for example. As shown in FIG. 2B, the system 210 may also include an analog signal output and one or more single and/or multi-parameter measurement monitors for obtaining, processing and/or relaying physiological and/or other data for a patient.

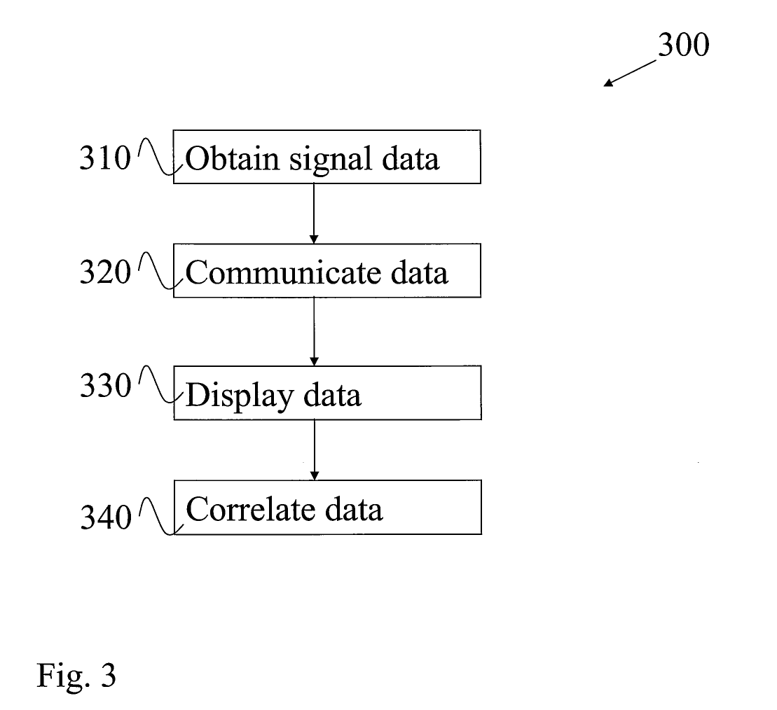

FIG. 3 illustrates a flow diagram for a method 300 for exchanging physiological signal data between an EP/Hemo recording/monitoring system and an ultrasound system in accordance with an embodiment of the present invention. At step 310, physiological signal data is obtained from a patient. For example, ECG and/or intracardiac waveform data is acquired from a patient by the EP/Hemo system using a sensor and/or other monitor.

At step 320, the physiological signal data is communicated from the EP/Hemo system to the ultrasound system via a data connection in an integrated EP/Hemo and ultrasound system environment. For example, ECG and/or intracardiac waveform data is transmitted from the EP/Hemo system to the ultrasound system via a wired and/or wireless signal data connection.

At step 330, the physiological signal data is displayed simultaneously (or at least substantially simultaneously given some inherent transmission and/or processing delay) at the EP/Hemo system and the ultrasound system. For example, a user at the EP/Hemo system and a user at the ultrasound system (and/or a user viewing both users) can see the transmitted physiological signal data concurrently. For example, while a practitioner is obtaining ultrasound images of a patient, the practitioner can also view an ECG for the patient.

At step 340, signal information is correlated with image information for display and/or processing. Thus, ECG/intracardiac waveform information may be correlated with ultrasound image data for display, further processing, reporting and/or analysis by a clinician, for example. For example, waveform data can be used to view specific image frames by specifying a given point in time directly on the waveform.

One or more of the steps of the method 300 may be implemented alone or in combination in hardware, firmware, and/or as a set of instructions in software, for example. Certain embodiments may be provided as a set of instructions residing on a computer-readable medium, such as a memory, hard disk, DVD, or CD, for execution on a general purpose computer or other processing device.

Certain embodiments of the present invention may omit one or more of these steps and/or perform the steps in a different order than the order listed. For example, some steps may not be performed in certain embodiments of the present invention. As a further example, certain steps may be performed in a different temporal order, including simultaneously, than listed above.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A method for exchanging physiological signal data between an electrophysiologic/hemodynamic (EP/Hemo) system and an ultrasound system in an integrated EP/Hemo and ultrasound system, said method comprising:
obtaining physiological signal data;
communicating said physiological signal data between said EP/Hemo system and said ultrasound system via signal communication ports;
displaying said physiological signal data at both of said EP/Hemo system and said ultrasound system.

2. The method of claim 1, further comprising correlating said physiological signal data with image data from said ultrasound system.

3. The method of claim 1, wherein said displaying step further comprises displaying said physiological signal data substantially simultaneously at both of said EP/Hemo system and said ultrasound system.

4. The method of claim 1, wherein said physiological signal data comprises waveform signal data.

5. The method of claim 4, wherein said waveform signal data comprises at least one of electrocardiogram waveform data and intracardiac waveform data.

6. The method of claim 1, wherein said communicating step comprises at least one of wired and wireless communication of said physiological signal data between said EP/Hemo system and said ultrasound system via signal communication ports.

7. The method of claim 1, further comprising using said waveform signal data to gate image capture at said ultrasound system.

8. The method of claim 1, wherein said communicating step further comprises communicating said physiological signal data between said EP/Hemo system and said ultrasound system via signal communication ports using an interface.

9. An integrated electrophysiologic/hemodynamic (EP/Hemo) and ultrasound system, said system comprising:
an EP/Hemo system for obtaining physiological signal data for a patient, said EP/Hemo system including a first signal communication port;
an ultrasound system for obtaining ultrasound data for said patient, said ultrasound system including a second signal communication port; and
a communication channel between said EP/Hemo system and said ultrasound system for communicating said physiological signal data between said EP/Hemo system and said ultrasound system via said first and second signal communication ports;
wherein said EP/Hemo system and said ultrasound system are configured for display of said physiological signal data at both of said EP/Hemo system and said ultrasound system.

10. The system of claim 9, wherein at least one of said ultrasound system and said EP/Hemo system correlates said physiological signal data with image data from said ultrasound system.

11. The system of claim 9, wherein said physiological signal data is displayed substantially simultaneously at both of said EP/Hemo system and said ultrasound system.

12. The system of claim 9, wherein said physiological signal data comprises waveform signal data.

13. The system of claim 12, wherein said waveform signal data comprises at least one of electrocardiogram waveform data and intracardiac waveform data.

14. The system of claim 9, wherein said communication channel comprises at least one of wired and wireless communication of said physiological signal data between said EP/Hemo system and said ultrasound system via said first and second signal communication ports.

15. The system of claim 9, wherein said ultrasound system uses said physiological signal data to at least one of gate image capture and perform image processing.

16. The system of claim 9, wherein at least one of said EP/Hemo system and system ultrasound system further comprises an interface for communicating said physiological signal data between said EP/Hemo system and said ultrasound system via said first and second signal communication ports.

17. A method for integrating physiological and imaging data for a patient, said method comprising:
providing physiological waveform data for a patient;
providing image data for the patient;
correlating said physiological waveform data and said image data for the patient; and
displaying said physiological signal waveform data and said image data in an integrated physiological and imaging environment.

18. The method of claim 17, further comprising communicating said physiological waveform data via a dedicated communication channel for correlation with said image data.

19. The method of claim 17, wherein said physiological waveform data comprises at least one of electrocardiogram and intracardiac waveform data.

20. The method of claim 17, wherein said imaging data comprises ultrasound imaging data.
